(19) United States
(12) Patent Application Publication
(10) Pub. No.: US 2025/0331749 A1
(43) Pub. Date: Oct. 30, 2025

(54) ANALYTE SENSOR

(71) Applicant: PercuSense, Inc., Valencia, CA (US)

(72) Inventors: Rajiv Shah, Rancho Palos Verdes, CA (US); Blake Morrow, Los Angeles, CA (US); Brian Kannard, Los Angeles, CA (US); Bradley Liang, Bloomfield Hills, MI (US); Kaitlynn Olczak, Valenica, CA (US); Katherine Wolfe, Dunwoody, GA (US)

(73) Assignee: PercuSense, Inc., Valencia, CA (US)

(21) Appl. No.: 19/263,263

(22) Filed: Jul. 8, 2025

Related U.S. Application Data
(63) Continuation of application No. PCT/US2023/080616, filed on Nov. 20, 2023.
(60) Provisional application No. 63/439,183, filed on Jan. 16, 2023.

(51) Int. Cl. A61B 5/1486; A61B 5/145; C12Q 1/00

(52) U.S. Cl. CPC A61B 5/1486; A61B 5/14532; C12Q 1/005; A61B 2562/182

(57) ABSTRACT

In one embodiment, an analyte sensor is disclosed. The analyte sensor includes a working conductor having an electrode reactive surface. The analyte sensor further includes a first reactive chemistry being responsive to a first analyte and a first transport matrix that includes a first transport material and a mitigation compound. The first transport material enables flux of the first analyte to the first reactive chemistry. The analyte sensor includes a second transport material disposed over and configured to enable transport of a reactant to the first reactive chemistry. Wherein the first reactive chemistry does not contact the electrode reactive surface while at least partially overlapping a portion of the electrode reactive surface.

### RELATED APPLICATIONS

This application claims the benefit of U.S. provisional application Ser. No. 63/439,183, filed on Jan. 16, 2023. The application listed above is hereby incorporated by reference in its entirety for all purposes.

### FIELD OF THE INVENTION

The present invention is generally directed to devices and methods that perform in vivo monitoring of an analyte or analytes such as, but not limited glucose, lactate or ketones. In particular, the devices and methods are for electrochemical sensors that provide information regarding the presence or amount of an analyte or analytes within a subject.

### BACKGROUND OF THE INVENTION

In vivo monitoring of particular analytes can be critically important to short-term and long-term well being. For example, the monitoring of glucose can be particularly important for people with diabetes in order to determine insulin or glucose requirements. In another example, the monitoring of lactate in postoperative patients can provide critical information regarding the detection and treatment of sepsis.

The need to perform continuous or near continuous analyte monitoring has resulted in the development of a variety of devices and methods. Some methods place electrochemical sensor devices designed to detect the desired analyte in blood vessels while other methods place the devices in subcutaneous or interstitial fluid. Both placement locations can provide challenges to receiving consistently valid data. Furthermore, achieving consistent placement location can be critical to hydrating, conditioning and calibrating the device before actual use. Hydrating and conditioning of commercially available sensor devices can be a time consuming process often taking fractions of hours up to multiple hours, to significant fractions of days. Assuming the hydrating and conditioning process is completed successfully, a user may have to compromise their freedom of movement or range of movement in order to keep the sensor properly located within their body.

Glucose sensors are one example of in vivo continuous analyte monitoring. Commercially available implantable glucose sensors generally employ electrodes fabricated on a planar substrate or wire electrodes. In either configuration the electrode surface is coated with an enzyme which is then further coated with a polymer membrane to control the amount of glucose and oxygen that reaches the electrode surface. In some glucose sensors the polymer membrane is hydrophilic which allows glucose to easily diffuse through the membrane layer, however the hydrophilic membrane severely limits the amount of oxygen that can diffuse through the membrane. The lack of oxygen on the electrode surface can become an issue because the glucose sensor works by using the enzyme to catalyze a reaction between glucose and oxygen resulting in hydrogen peroxide that is oxidized at a working electrode. Only when there is an abundance of oxygen present at the working electrode, will the glucose measured by the electrode be proportional to the amount of glucose that reacts with the enzyme. Otherwise, in instances where insufficient oxygen is present at the working electrode, the glucose measurement is proportional to the oxygen concentration rather than the glucose concentration.

Further exacerbating the problem is the deficiency of oxygen relative to glucose in the human body. The ratio of glucose to oxygen in the human body ranges from approximately 10-to-1 to 1000-to-1. This typically means the enzyme catalyzed reaction at the working electrode is generally operating in a condition of oxygen deficiency which can result in many critical problems that influence accuracy, sensitivity and long-term reliability of in vivo sensors. Various approaches have been implemented to counteract the oxygen deficiency problem and increase the relative concentration of available oxygen at the electrode. For example, commercially available glucose sensor systems rely on a highly specialized glucose limiting membrane (GLM) rather than the simply hydrophilic membrane discussed above. Multiple commercial approaches have GLMs that are homogeneous membranes with both hydrophobic and hydrophilic regions to draw in oxygen while also drawing in glucose. One drawback to the implementation of GLMs is the increased cost of the sensor due to the increased cost to manufacture the complex GLMs. Furthermore, material variability within the GLM and non-uniform dispersion of the hydrophilic areas often result in batch to batch variability that affects accuracy, sensitivity and reliability of the sensor. Additionally, because of the hydrophilic and hydrophobic areas of the GLM, diffusion of either glucose or oxygen occurs primarily perpendicular to the surface of the electrode.

Another drawback associated with the use of GLM is that effectiveness of a sensor may be adversely affected if metabolically active cells associated with insertion site trauma or host response interferes with or blocks a portion of the GLM. For example, if red blood cells were to pool in close proximity to the GLM, flow of glucose and oxygen to the sensor electrode could be significantly impeded. Similarly, if white blood cells obstructed flow of glucose across the hydrophilic areas of a GLM the sensor electrode would output erroneous data because glucose that should otherwise reach the working electrode is being consumed by the white blood cells and there is no alternative path for glucose to diffuse to the working electrode.

Another drawback is the hydrophobic nature of GLM. The use of GLM can at least partially explain prolonged hydration and conditioning time for many commercially available glucose sensors. Hydration and conditioning of the sensor requires transportation of fluid to the working electrode. However, because GLM favors the transport of oxygen, the hydrophobic regions of the GLM are placed over the electrode to promote diffusion of oxygen to the electrode. Being hydrophobic, those same areas repel water that is necessary to hydrate the sensor and transport the glucose to the electrode.

The claimed invention seeks to address many of the issues discussed above regarding in vivo monitoring of particular analytes. In many examples discussed below, the analyte being measured is glucose. In still other examples the analyte is lactate. However, while specific embodiments and examples may be related to glucose or lactate, the scope of the disclosure and claims should not be construed to be limited to either glucose or lactate. Rather it should be recognized that the chemistry applied to the electrodes of the sensors described herein is determinative of the analyte the sensor measures.

### BRIEF SUMMARY OF THE INVENTION

In one embodiment, an analyte sensor is disclosed. The analyte sensor includes a working conductor having an electrode reactive surface. The analyte sensor further includes a first reactive chemistry being responsive to a first analyte and a first transport matrix that includes a first transport material and a mitigation compound, the first transport material enables flux of the first analyte to the first reactive chemistry. Additionally included in the analyte sensor is a second transport material disposed over and configured to enable transport of a reactant to the first reactive chemistry. Wherein the first reactive chemistry does not contact the electrode reactive surface while at least partially overlapping a portion of the electrode reactive surface.

In another embodiment, a method to expose an electrical conductor that is encapsulated within an electrical insulator is disclosed. The method includes the operation of
removing electrical insulator over a portion of a top of the electrical conductor to create a window within the electrical insulator, the window having a window edge, the window edge overlapping a portion of the top of the electrical conductor.

In still another embodiment, a working electrode within an electrochemical sensor assembly is disclosed. The working electrode includes a multilayer structure having an A-side and a B-side. The A-side includes a first insulation layer, a conductive layer adjacent to the first insulation layer and a via that traverses through the multilayer structure from the A-side to the B-side. The A-side further having a first reactive chemistry disposed over and in contact with a portion of the first insulation layer and the first reactive chemistry further being in disposed over and in contact with the conductive layer and partially filling the via to define a reactive via having a reactive area. The B-side of the working electrode includes a second insulation layer, the via traverses through the second insulation layer and a second reactive chemistry partially fills the via through the second insulation layer from the B-side. Wherein the first reactive chemistry within the via prevents the second reactive chemistry from being in contact with the conductive layer.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIGS. 1A and 1B are exemplary illustrations of a distal end of a sensor having an A-side and a B-side, respectively, in accordance with embodiments of the present invention.

FIG. 2A is an exemplary illustration of a cross-section A-A of the sensor in FIGS. 1A and 1B in accordance with embodiments of the present invention.

FIG. 2B is an exemplary illustration of an alternative embodiment of cross-section A-A of the sensor in FIGS. 1A and 1B in accordance with embodiments of the present invention.

FIGS. 3A-3E are exemplary illustrations of various embodiments of working electrode configurations similar to those discussed in FIGS. 2A and 2B above, in accordance with embodiments of the present invention.

FIGS. 4A-4E are exemplary illustrations of different preparations that expose portions of either the working conductor or the counter reference conductor, in accordance with various embodiments of present invention.

FIGS. 5A-5F are exemplary illustrations of embodiments of a working electrode that includes an aperture through the working conductor, in accordance with embodiments of the present invention.

FIGS. 6A and 6B are exemplary illustrations of a sensor configured with sensing and non-sensing electrodes in order to control mass transfer gradients in diffusion fields primarily parallel to the electrode surface, in accordance with embodiments of the present invention.

### DETAILED DESCRIPTION

Presented below are embodiments of sensor configuration that is intended to enable continuous real-time in-vivo electrochemical sensing of an analyte or molecule, or analytes or molecules of interest within a subject. The in-vivo measurement within a subject is typically performed in tissue such as, but not limited to subcutaneous tissue. However, various embodiments can be inserted into the vasculature, musculature, or organ tissue. The sensor may include a working electrode along with a counter electrode and a reference electrode. Alternatively, many embodiments utilize a working electrode in conjunction with a combined counter/reference electrode.

Embodiments of the sensor can be configured to measure analytes such as lactate, ketones, glucose and the like. Furthermore, while some embodiments may be configured to measure a single or individual analyte, other embodiments can be configured to measure multiple analytes including various combinations of at least two or more molecules of interest such as lactate, ketone, glucose, oxygen, reactive oxygen species and the like. In still other embodiments, the sensors may be configured with infusion sets to enable sensing of a single or multiple molecule of interest while also enabling delivery of an infusate from a single point of entry.

Many commercially available real-time continuous implantable sensors, such as, but not limited to glucose sensors, are susceptible to interference from compounds such as, but not limited to acetaminophen, ascorbic acid, uric acid, and salicylic acid. The presence of a single or multiple interferent compounds can negatively impact sensor performance. A non-limiting example of a negative impact on sensor performance includes, but is not limited to, influencing or augmenting sensor data so that is no longer representative of the concentration of the analyte or molecule of interest within the subject.

Electrochemical sensors often rely on enzymes such as oxidase or dehydrogenase enzymes that are selected to react with the molecule of interest. Non-limiting examples include glucose oxidase to react with glucose, lactate oxidase to react with lactate, and 3-hydroxybutyrate dehydrogenase (3HBDH) to react with ketones. In the presence of the analyte of interest, the selected enzyme typically eventually generates hydrogen peroxide, NADP(H) or NAD(H) that is subsequently decomposed on the working electrode and the resultant electrical current is detected via some combination of a counter electrode, reference electrode, or combined counter/reference electrode.

In preferred embodiments, the generation/measurement of electrical current generated by the enzymatic reaction maintains a linear relationship with the concentration of the analyte of interest in the subject. However, in many embodiments, interferents such as acetaminophen may be decomposed on the working electrode along with the hydrogen peroxide generated by the enzymatic reaction from the analyte of interest. Accordingly, the presence of an interferent or interferents can result in current generation that is not proportional to the concentration of the analyte of interest within the subject.

A common trait among many interferent compounds is that they are negatively charged. Accordingly, the selective and purposeful inclusion of a mitigation compound having a negative charge within a sensor assembly may reduce the impact of interferent compounds by electrostatically repelling negatively charged interferent compounds. The repulsion of interferent compounds via the purposeful placement of a mitigation compound can encumber, delay or prevent the interferent compound from being able to negatively impact sensor performance.

In some embodiments, in addition to being negatively charged, it may be desirable for a mitigation compound to also be a relatively large molecule relative to the interference compound(s) and also relative to any other molecule within the matrix that includes the mitigation compound. The relatively large size of the mitigation compound can minimize the likelihood of migration of movement of the mitigation compound within the matrix surrounding the mitigation compound.

The various embodiments discussed below are intended to be exemplary and should not be viewed or construed as discrete individual embodiments. Rather, where possible, individual features or elements discussed in each embodiment should be considered transferable or combinable with the various other embodiments disclosed below.

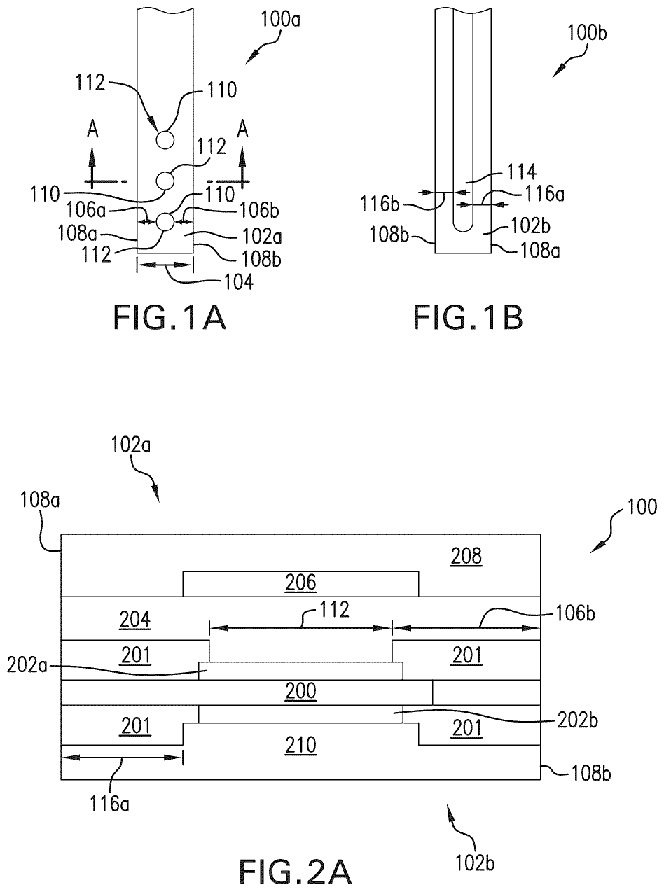

FIGS. 1A and 1B are exemplary illustrations of a distal end of a sensor assembly having an A-side 100a and a B-side 100b, respectively, in accordance with embodiments of the present invention. The sensor has a sensor width 104 that is defined between sensor edges 108a and 108b. The sensor illustrated in FIGS. 1A and 1B is intended to be implanted within a subject thereby placing the distal end of the sensor within bodily fluid containing an analyte or analytes of interest. In preferred embodiments, the distal end of the sensor is configured to electrochemically detect the presence of the analyte or analytes of interest. For simplicity, FIGS. 1A and 1B focus on the distal end of the sensor but it should be understood that the sensor configurations discussed throughout this disclosure include electrical contact pads that enable the operation of a two or three electrode system located in the distal end of the sensor.

FIG. 1A is an exemplary illustration of the A-side 100a that includes at least one working electrode 110 having a plurality of openings 112. The openings 112 are offset from the sensor edges 108a and 108b by working offsets 106a and 106b, respectively. In some embodiments, the working offsets 106a and 106b are substantially equal thereby locating the openings 112 substantially along a centerline of the sensor. In other embodiments, the openings 112 may be biased toward either sensor edge 108a or 108b. The openings 112 are illustrated as being substantially circular. However, in other embodiments the openings 112 may be any variety of shape or shapes. Moreover, the use of three openings 112 should not be construed as limiting. Various embodiments may use more or fewer openings 112 based on a variety of parameters such as, but not limited to, desired signal intensity or strength, enzyme activity, surface area and the like.

FIG. 1B is an exemplary illustration of the B-side 100b that is opposite the A-side 100a and includes at least one combined counter-reference electrode (CRE) 114. The CRE 114 is offset from the sensor edges 108a and 108b by CRE offsets 116a and 116b respectively. In some embodiments, the CRE offsets 116a and 116b are substantially equal thereby centering the CRE 114 between sensor edges 108a and 108b. The use of the combined CRE 114 should not be construed as limiting. The inclusion of an additional electrical conductor enables the use of a three electrode system having a separate counter electrode and reference electrode. Moreover, in embodiments utilizing a three electrode system (separate electrodes for each of working, counter and reference) two of the three may be placed on either A-side 102a or B-side 102b, while the remaining electrode is placed on the opposite side. In still other embodiments of a three electrode system, all three electrodes are formed or placed on either the A-side 102a or the B-side 102b of the sensor assembly.

Additionally, FIGS. 1A and 1B should not be construed as being proportional or representative of relative dimensions within a sensor. For example, in many embodiments the sensor width 104 falls between a range of 0.005 inches and 0.1 inches. Additionally, the opening 112 may be formed to be within a range between 0.0002 inches and 0.03 inches. Similarly, in many embodiments the CRE 114 is formed to have a width between 0.002 inches and 0.03 inches. In many embodiments a CRE opening 114′ is substantially the same size as the CRE 114. In other embodiments, the CRE opening 114′ is larger or smaller than the CRE 114.

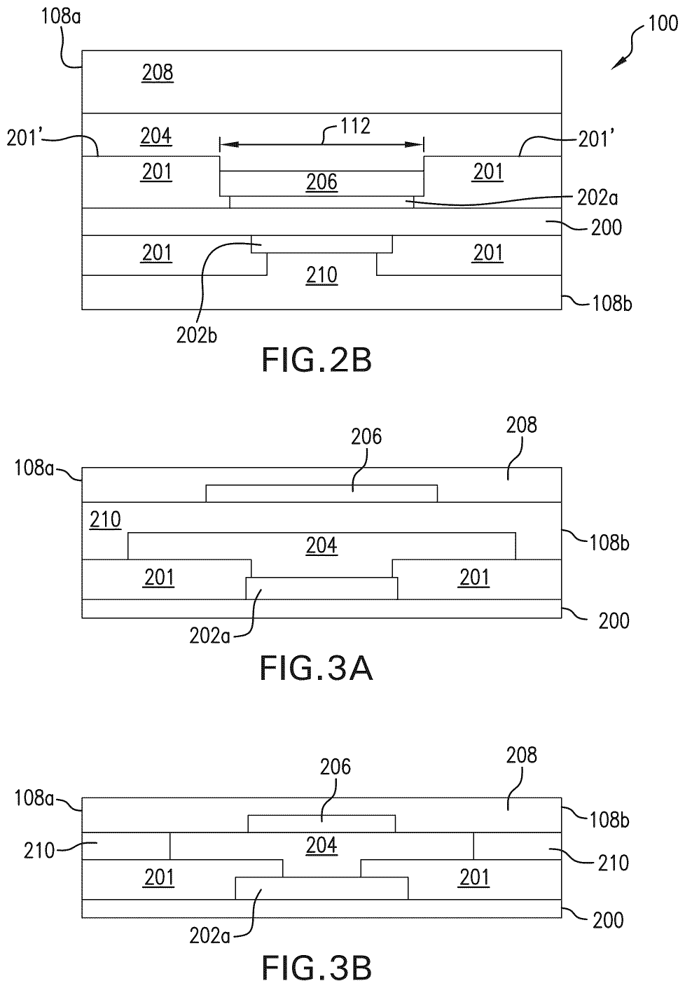

FIG. 2A is an exemplary illustration of a cross-section A-A of the sensor in FIGS. 1A and 1B in accordance with embodiments of the present invention. Insulation 200 electrically isolates a working conductor 202a from a combined counter-reference conductor (CRC) 202b. In preferred embodiments, the working conductor 202a and CRC 202b are coupled to the insulation 200. An exemplary, non-limiting coupling technique includes adhesives to couple either or both of the working conductor 202a and CRC 202b to the insulation 200. On A-side 100a, insulation 201 has opening 112, but otherwise covers both the insulation 200 and the working conductor 202a. The opening 112 exposes a portion of the working conductor 202a. On the B-side, insulation 201 has an opening for the CRE 114 where insulation 201 covers the insulation 200 up to the CRC 202b so the opening in insulation 201 exposes the CRC 202b. In many embodiments, the insulation 201 is coupled to the insulation 200 and at least one or both of the working conductor 202a and the CRC 202b. A non-limiting, exemplary method such as an adhesive may be used to couple the insulation 201 to insulation 200 and one or more of the working conducted 202a and the CRC 202b.

Continuing with the A-side 102a, a first transport mix 204 is located over the insulation 201 and the exposed working conductor 202a. The first transport mix 204 fills the opening 112 and extends contiguously from the sensor edge 108a across the sensor width 104 to the sensor edge 108b. In preferred embodiments, the first transport mix 204 is a combination or mixture of a first transport material and a mitigation compound. In preferred embodiments, the first transport material is a hydrogel that is selected based on its capacity to freely enable transport or movement of the analyte of interest found within the tissue surrounding the sensor. For example, if the first transport material was immersed in interstitial fluid containing the analyte of interest, eventually the concentration of the analyte of interest within and throughout the first transport material would be identical to the concentration within the interstitial fluid. Because the first transport material is selected based on its ability to freely transport or move the analyte or analytes of interest, interferent compounds may also be free to move within and throughout the first transport material. As discussed above, interferent compounds reaching the working conductor 202a can result in electrical current generation that is not proportional to the concentration of the analyte of interest within the bodily fluid surrounding the sensor.

To reduce or minimize the movement of interferent compounds within the first transport mix 204, a mitigation compound is blended, mixed or combined with the first transport material. For example, in some embodiments albumin or similar compounds may be combined or included with the different transport materials or within the reactive chemistry. In preferred embodiments, the mitigation compound is selected based on its ability to minimize or reduce the transmission of an interferent compound through the first transport material. The mitigation compound may be selected based on characteristics such as, but not limited to properties such as electrical charge and the relative size of the mitigation compound (e.g., molecular size). Selecting the mitigation compound based on electrical charge enables repulsion of interferent compounds having a like electrical charge. For example, acetaminophen is often considered an undesirable interferent compound that has a negative electrical charge. Accordingly, inclusion of a mitigation compound that has a negative electrical charge can reduce or minimize movement or transport of acetaminophen within the first transport material.

In some embodiments, the mitigation compound includes a macromolecule that is electrically charged or charged but electrically neutral. In other embodiments, the mitigation compound includes pendant and covalently bonded molecules that are charged or charged but electrically neutral. In still other embodiments the mitigation compound is a combination of macromolecules and pendant and covalently bonded molecules. In many embodiments, molecular size may also be considered when selecting the mitigation compound. The molecular size can be selected based on the chemical nature of the first transport material depending on whether the charged entities are immobilized or covalently bound to the first transport material.

Located on top of the first transport mix 204 are both a second transport material 208 and a reactive chemistry 206. In preferred embodiments, the reactive chemistry 206 includes an enzyme selected to react with the analyte of interest. Exemplary enzymes that may be used within the reactive chemistry 206 include, but are not limited to, oxidases and dehydrogenases such as glucose oxidase, glucose dehydrogenase, lactate oxidase, lactate dehydrogenase, and 3-hydroxybutyrate dehydrogenase. Additionally, in some embodiments, coenzyme elements such as, but not limited to NAD, NADP, and FAD may be included in the reactive chemistry. In many embodiments, the reactive chemistry 206 is a mixture of an enzyme and polymer that is located and cured over a portion of the first transport mix 204.

As illustrated in FIG. 2A, the reactive chemistry 206 is directly in contact with the first transport mix 204 and overlaps the opening 112. The first reactive chemistry 206 does not extend to either of the sensor edges 108a and 108b. Because the first transport mix 204 is positioned between the opening 112 and the reactive chemistry 206, the reactive chemistry 206 can be viewed as completely or entirely eclipsing the opening 112. In alternative embodiments the reactive chemistry 206 may not overlap the opening 112, or may be sized to be smaller than the opening 112. In these embodiments the reactive chemistry 206 may be viewed as partially eclipsing the opening 112.

The second transport material 208 is located on top of both the first transport mix 204 and the reactive chemistry 206. The second transport material 208 extends contiguously between sensor edges 108a and 108b. In many embodiments, the second transport material 208 is impervious to the analyte of interest. In these embodiments, the analyte of interest is able to enter the sensor through the first transport mix 204 exposed along the sensor edges 108a and 108b. In embodiments where the analyte of interest is glucose, silicone would be an exemplary second transport material 208.

Returning to the B-side 102b in FIG. 2A, a third transport material 210 may be applied over both the insulation 201 and the exposed CRC 202b. As illustrated in FIG. 2A, the third transport material 210 may be applied contiguously from the sensor edge 108a across the sensor width 104, to the sensor edge 108b. In alternative embodiments, the second transport material 210 may be applied continuously between the sensor edges 108a and 108b, while not extending up to the sensor edges 108a and 108b. In many embodiments the third transport material 210 is identical to the first transport material used within the first transport mix 204. In other embodiments, the third transport material 210 is selected based on criteria such as, but not limited to biocompatibility and being transmissive or transparent to electrical current.

FIG. 2B is an exemplary illustration of an alternative embodiment of cross-section A-A of the sensor in FIGS. 1A and 1B in accordance with embodiments of the present invention. In the embodiment illustrated in FIG. 2B the reactive chemistry 206 is located directly in contact with the working conductor 202a. The reactive chemistry 206 is contiguously applied over an entirety of the exposed working conductor 202a but the reactive chemistry 206 does not entirely fill the openings 112. Specifically, the reactive chemistry 206 does not reach an insulation top 201′. However, in various other embodiments, the reactive chemistry 206 may be applied within the opening 112 to be even with or even spill over and onto, the insulation top 201′.

In embodiments of both FIGS. 2A and 2B, the first transport mix 204 exposed along the sensor edges 108a and 108b is the primary conduit or pathway for fluid surrounding the sensor to enter the sensor.

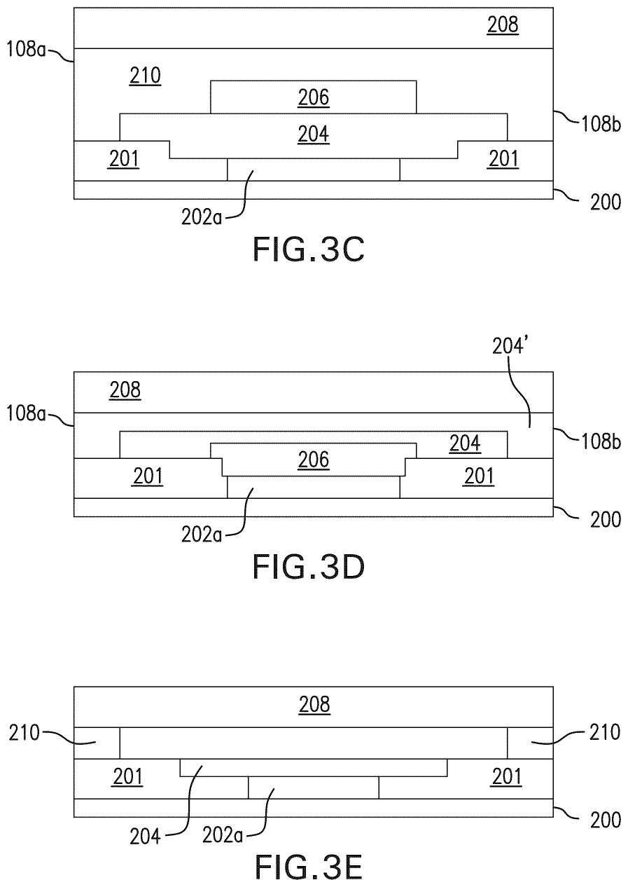

FIGS. 3A-3E are exemplary illustrations of various embodiments of working electrode configurations similar to those discussed in FIGS. 2A and 2B above, in accordance with embodiments of the present invention. FIG. 3A is an embodiment where the reactive chemistry 206 is not directly in contact with the working conducted 202a and the first transport mix 204 does not extend to the sensor edges 108a and 108b. Additionally, a third transport material 210 encapsulates the first transport mix 204. Moreover, the third transport material 210 is located or placed as a continuous layer between the sensor edges 108a and 108b. Furthermore, the third transport material 210 is in contact with the insulation 201, the first transport mix 204, the second transport material 208 and the reactive chemistry 206.

In many embodiments the third transport material 210 is selected based on its ability to enable unencumbered transport or movement of fluid and any analyte or analytes of interest therein, that is in contact with the sensor edges 108a and 108b. A non-limiting, exemplary third transport material 210 is a hydrogel. Accordingly, the third transport material 210 is capable of enabling transport, movement or diffusion of interferent compounds found within fluid surrounding the sensor to an interior of the sensor. However, the ability of any interferent compounds to reach the working conductor 202a is minimized because the first transport mix 204 that includes the mitigation compound is placed directly over the working conductor 202a.

FIG. 3B is another non-limiting, exemplary embodiment where the reactive chemistry 206 is not directly in contact with the working conductor 202a and the first transport mix 204 does not extend to the sensor edges 108a and 108b. In FIG. 3B the third transport material 210 is directly in contact with the insulation 201, the second transport material 208 and the first transport mix 204. Note that the third transport material 210 has an exposed face along sensor edges 108a and 108b. However, in FIG. 3B, the third transport material 210 does not extend over and across the first transport mix 204.

FIG. 3C is still an additional non-limiting, exemplary embodiment where the reactive chemistry 206 is not directly in contact with the working conductor 202a and the first transport mix 204 does not extend to the sensor edges 108a and 108b. In FIG. 3C, the third transport material 210 is directly in contact with the insulation 201, the first transport mix 204, the reactive chemistry 206 and the second transport material 208. In FIG. 3C, the reactive chemistry 206 is directly in contact with the first transport mix 204 and is not directly in contact with the second transport material 208. Additionally, the third transport material 210 does form a contiguous layer spanning the width of the sensor and further separates the second transport material 208 from the other components within the sensor.

FIG. 3D is a non-limiting, exemplary embodiment of a sensor where the reactive chemistry 206 is directly in contact with the working conductor 202a and the first transport mix 204 does not extend to the sensor edges 108a and 108b. In FIG. 3D, the reactive chemistry 206 is directly in contact with the working conductor 202a and the first transport mix 204 encapsulates or covers the reactive chemistry 206. Additionally, the first transport mix 204 does not span the width of the sensor and accordingly does not extend to the sensor edges 108a and 108b. Rather, the third transport material 210 is located at the sensor edges 108a and 108b. The third transport material 210 extends across the sensor while being directly in contact with the insulation 201, the first transport mix 204 and the second transport materials 208.

FIG. 3E is a non-limiting, exemplary embodiment of a sensor that does not include the reactive chemistry 206. Without the reactive chemistry 206, the working conductor 202a can be used to detect oxygen concentrations. In the embodiments shown in FIG. 3E, the first transport mix 204 is applied over the working conductor 202a to prevent interferent compounds from reaching the working conductor 202a. In FIG. 3E the first transport mix 204 does not extend to the sensor edges 108a and 108b. Located at the sensor edges 108a and 108b is the third transport material 210. The third transport material 210 is directly in contact with the insulation 2210, the

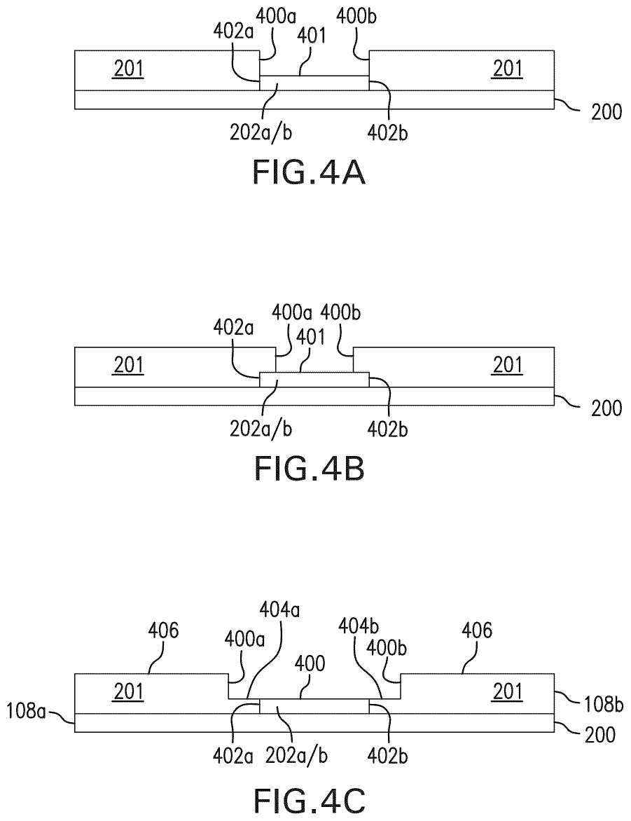

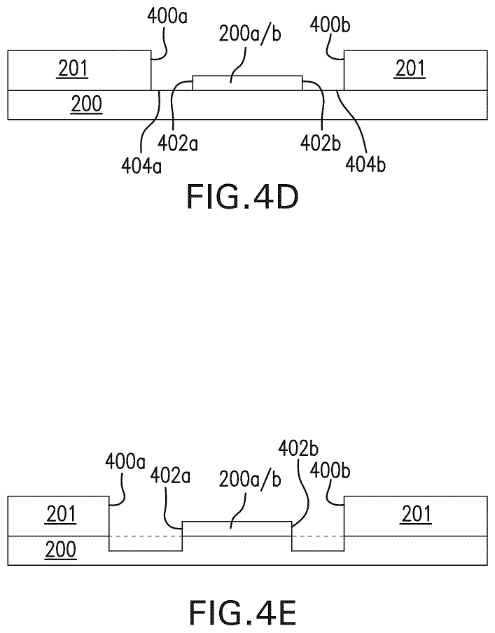

FIGS. 4A-4E are exemplary illustrations of different preparations that expose portions of either the working conductor 202a or the CRC 202b, in accordance with various embodiments of present invention. To form the working electrode 110, the insulation 201 includes openings 112 that selectively expose the working conductor 202a. Similarly, to form the CRE 114, an opening is formed in the insulation 201 to selectively expose an area of the CRC 202b. As the preparations discussed across FIGS. 4A-4E may be applied to either or both the working conductor 202a and the CRC 202b, for simplicity they will be referred to as the conductor 202a/202b or the conductors 202a/202b.

Selective removal of the insulation 201 exposes various portions of the conductors 202a/202b while also creating features within the insulation 201. For example, in FIG. 4A, conductor 202a/202b has conductor edges 402a and 402b. Similarly, the insulation 201 has window edges 400a and 400b. In FIG. 4A, the windows edges 400a and 400b are formed to be substantially aligned with the conductor edges 402a and 402b. This results in the entirety of a top 401 of the conductor 202a/202b being exposed.

FIG. 4B is an exemplary illustration of an alternate embodiment where only a portion of the top 401 of the conductor 202a/202b is exposed. In FIG. 4B, the window edges 400a and 400b overlap the top 401 of the conductor 202a/202b. Alternatively, the conductor edges 402a and 402b are tucked under the window edges 400a and 400b. In embodiments where less area of the conductor 202a/202b needs to be exposed, it may be advantageous to have the insulation 201 overlap a portion of the conductor 202a or 202b in order to simplify the manufacturing process or tune or change stiffness properties of the sensor assembly.

FIG. 4C is an exemplary illustration of an embodiment where the window 112 is larger, or expands beyond the conductor 202a/202b. In this embodiment the window edges 400a and 400b are defined between an insulation top 406 and an insulation recess 404a and 404b. In many embodiments the window edges 400a and 400b, along with the insulation recesses 404a and 404b are created by removing the insulation 201 to a preferred depth. As illustrated in FIG. 4C, the insulation recesses 404a and 404b are substantially even with the top 401 of the electrode 202a/202b. Additionally, the insulation recesses 404a and 404b extend away from the electrode 202a/202b toward the edges 108a and 108b. Partial removal of the insulation 201 may be accomplished using a variety of techniques such as, but not limited to laser ablation, lithography or the like. The increased area formed by the insulation recesses 404a and 404b in proximity to the top 401 of the electrode 202a/202b can enable increased surface area for the propagation of surface preparation such as, but not limited to, plating of the electrode 202a/202b.

FIG. 4D is another exemplary illustration of an embodiment where the window 112 exposes the conductor 202a/202b, including conductor edges 402a and 402b. In this embodiment the insulation 201 is removed in order to expose at least a portion of the conductor edges 402a and 402b. In some embodiments, an entirety of the conductors edges 402a and 402b are exposed. Similar to the embodiments in FIG. 4C, exposure of the conductor edges 402a and 402b increases the surface area of the conductor 202a/202b that can support propagation of surface preparation techniques such as, but not limited to plating. The embodiments illustrated in FIGS. 4C and 4D should not be construed as limiting. In various other embodiments the conductor edges 402a and 402b may be partially exposed.

FIG. 4E is still another exemplary illustration of an embodiment where the window edges 400a and 400b are formed at a depth that intrudes into insulation 200. Because the conductor 202a/202b are coupled to insulation 200, in FIG. 4E, the insulation recesses 404a and 404b are formed below the conductor edges 402a and 402b. By removing additional insulation 200, this embodiment can provide even more additional surface area for the promotion or propagation of surface preparation techniques such as plating. In addition to plating, the wells or depressions created with the larger or wider insulation opening create larger volumes that can be filled with reactive chemistry or other materials. Across FIGS. 4A-4E it should be noted that the window 112 is shown as being substantially symmetrical in each different figure. However, in some embodiments various combinations of the embodiments shown in different figures may be combined on a single electrode or electrodes.

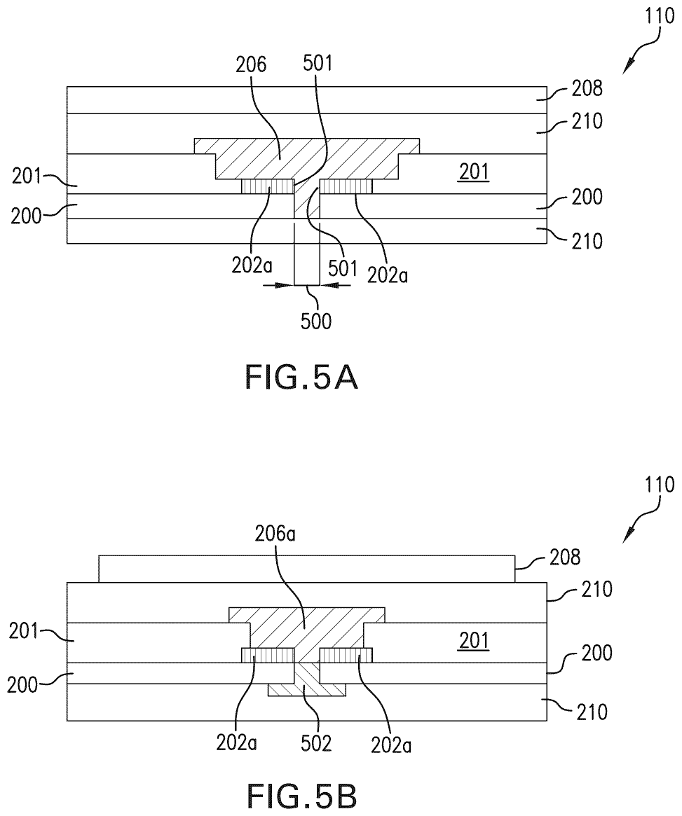

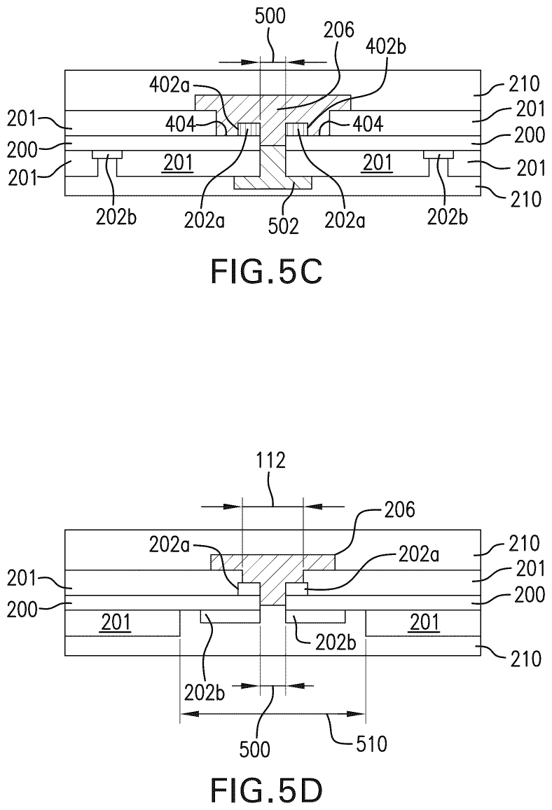

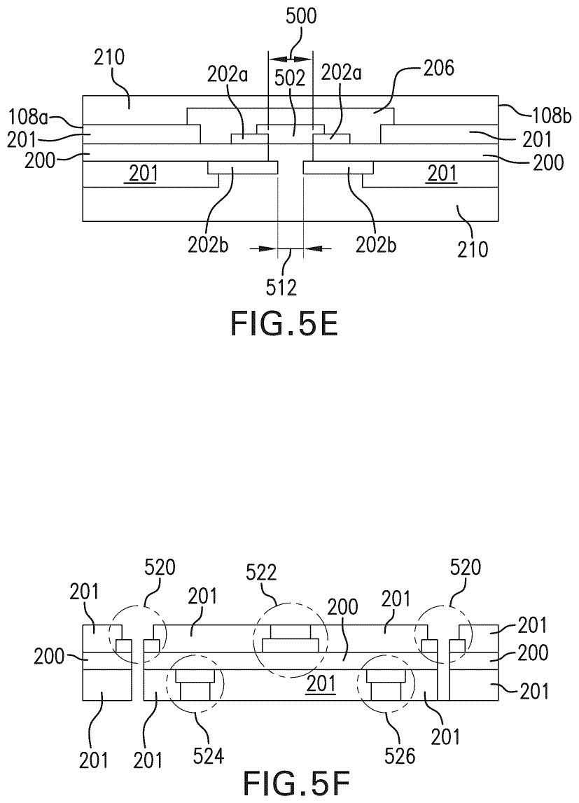

FIGS. 5A-5F are exemplary illustrations of embodiments of a working electrode that includes an aperture 500 through the working conductor 202a, in accordance with embodiments of the present invention. The aperture 500 includes an edge 501 formed in and completely through the working conductor 202a. An alternative name for the edge 501 is a lip or an opening. The edge 501 provides a unique surface that allows or enables differentiated sensor specific response due to tailored materials and processes applied on either side of the working conductor 202a. In some embodiments, the combination of inert materials and biorecognition elements such as enzymes, antibodies, aptamers and other chemical or biological recognition elements may be applied to either side of the aperture electrode to enable both detection of molecules of interest and regeneration of molecules to support the molecule of interest being detected. For example, in some embodiments enzyme cofactor recycling or regeneration are enabled by the selective application and placement of inert elements, enzymes, and other materials.

FIG. 5A is an exemplary illustration of a two-sided working electrode having an aperture 500 through the working conductor 202a. In some alternative embodiments, a second portion of the working conductor is placed on the second side of the sensor assembly and electrically connected through the aperture. In such embodiments, the sensor features an increased surface area within a similar sensor probe dimension. In FIG. 5A the aperture 500 is formed through the working conductor 202a along with insulation 200, thereby enabling access through either side of the working electrode 110. The reactive chemistry 206 is directly in contact with the working conductor 202a and extends through the aperture 500 and the insulation 200. A third transport material 210 extends across the sensor from sensor edge 108a to sensor edge 108b while also covering the reactive chemistry 206. In many embodiments the third reactive chemistry is a hydrogel that enables fluid surrounding the sensor to be transported in a substantially unencumbered manner across and through the sensor. An optional second transport material 208 is applied over the third transport material 210. In FIG. 5A the second transport material 208 is applied from sensor edge 208a to sensor edge 208b. However, in other embodiments, the second transport material 208 does not extend one or both of the sensor edges 208a and 208b. An optional layer of third transport material 210 is applied over the insulation 200. This configuration enables the analyte of interest to reach the reactive chemistry 206 via the edges 108a and 108b as well as through the aperture 500. The configuration in FIG. 5A does not include a cofactor.

FIG. 5B is an alternative embodiment of the working electrode 110 utilizing an aperture 500 that has a first reactive chemistry 206a and the second reactive chemistry 502. The embodiments in FIG. 5B may be particularly useful in embodiments where the first reactive chemistry 206a is a dehydrogenase based enzyme and the second reactive chemistry 502 includes a cofactor that is necessary for the dehydrogenase based enzyme to react with the analyte of interest. This embodiment diffuses the molecule of interest to a high surface area working conductor 202a with an aperture that has a dehydrogenase based enzyme immobilized on the bulk and surface of a structure that coats one side of the aperture electrode where a portion of the immobilized enzyme protrudes through the aperture but does not coat the opposite side of the aperture electrode. On the side opposite the enzyme, a mobile version of the enzyme cofactor or polymer with attached cofactor that is entrapped without substantially impacting cofactor mobility is able to interact with the immobilized dehydrogenase enzyme and can be recycled or regenerated through redox reactions on the electrode adjacent to the immobilized dehydrogenase enzyme. This enables the sensor to be continually operated with the magnitude of current required for cofactor recycling being related to the bulk concentration of the molecule of interest that reacts with the dehydrogenase enzyme.

The first reactive chemistry 206a is placed directly in contact with the working conductor 202a. In many embodiments the first reactive chemistry 206a is a dehydrogenase based enzyme that is immobilized on the working conductor that includes an aperture. Additionally, the first reactive chemistry 206a protrudes into the aperture 500, but does not extend through the aperture 500 to the opposite side. On the opposite side of the first reactive chemistry 206a is the second reactive chemistry 502. The second reactive chemistry 502 is directly in contact with the first reactive chemistry 502 within the aperture 500. In this embodiment, the second reactive chemistry 502 includes a cofactor to the dehydrogenase enzyme incorporated into the first reactive chemistry 206a. In preferred embodiments the cofactor remains mobile in the second reactive chemistry 502. In some embodiments the cofactor is a polymer with attached cofactor that is entrapped within a matrix of second reactive chemistry 502 that enables interaction of the cofactor with the immobilized dehydrogenase enzyme within the first reactive chemistry 206a.

Similar to FIG. 5A, a third transport material 210 spans the sensor and covers the first reactive chemistry 206a. An optional second transport material 208 covers the third transport material 210. In embodiments that include the second transport material 208, the analyte of interest may be limited or restricted to entering the sensor via the sensor edges 108a and 108b. In embodiments that do not include the second transport material 208, the analyte of interest is able to be freely transported to via every portion of the third transport material 210 across the sensor. In many embodiments, the third transport material 210 covers at least the second reactive chemistry 502. In FIG. 5B, the third transport material 210 extends from sensor edge 108a to sensor 108b while also covering the second reactive chemistry 502. However, in other embodiments, it is not necessary for the third transport material 210 to extend from sensor edge 108a to sensor edge 108b on the side opposite the first reactive chemistry 206a. In still other embodiments, the second reactive chemistry 502 is not covered by the optional third transport material 210.

FIG. 5C is an exemplary embodiment of a sensor assembly having a working conductor 202a that includes an aperture 500 and a CRC 202b, in accordance with embodiments of the present invention. This embodiment illustrates a configuration of a two electrode sensor where the working electrode is formed on the working conductor 202a and a combined counter reference electrode is formed on the CRC 202b. The two electrode system illustrated in FIG. 5C should not be construed as limiting as the design can be adapted or modified to function with a working electrode, counter electrode and reference electrode.

In FIG. 5C the working electrode is formed similar to the embodiment described in FIG. 5B. However, at least one difference between FIG. 5C and FIG. 5B is that the working conductor 202a and insulation 201 has been prepared similar to the embodiment illustrated in FIG. 4D. The exposure of conductor edges 402a and 402b increase the surface area of the working conductor 202a. Additionally, exposure of insulation recess 404 creates additional volume and area for the reactive chemistry 206. The additional exposure of the working conductor 202a to a larger volume or quantity of the reactive chemistry 206 enables improved sensor performance such as, but not limited to improved sensitivity to the analyte of interest, improved current output and improved sensor lifespan. Furthermore, the inclusion of second reactive chemistry 502 enables the embodiment in FIG. 5C to be used with dehydrogenase based enzymes. Similar to FIG. 5C, the second reactive chemistry 502 is located within the aperture 500 and provides cofactor that is complementary to a reaction between the analyte of interest and the first reactive chemistry 206. In some embodiments, the first reactive chemistry 206 is a dehydrogenase based enzyme such as, but not limited to glucose dehydrogenase, lactate dehydrogenase, or 3-hydroxybutyrate dehydrogenase. In these embodiments, the complementary second reactive chemistry 502 may be a cofactor, such as, but not limited to NAD or NAD+.

FIG. 5D is an exemplary sensor assembly that includes a working conductor 202a with an opening 112 and a combined counter-reference conductor 202b with an opening 510 and the aperture 500 is formed through both the working conductor 202a and the CRC 202b, in accordance with embodiments of the present invention. In this embodiment, the working conductor 202a and the CRC 202b have different widths. Additionally, the opening 112 associated with the working conductor 202a and the opening 510 associated with the CRC 202b have different widths. Moreover, FIG. 5D illustrated that the working conductor 202a and the CRC 202b may have different electrode preparations as discussed in FIGS. 4A-4E. It should be noted that with the aperture 500 going through both the working conductor 202a and the CRC 202b it may be preferable to optionally apply or omit the second reactive chemistry.

FIG. 5E is an exemplary sensor assembly that includes a working conductor 202a with an aperture 500 and a combined counter-reference conductor 202b with a second aperture 512, in accordance with embodiments of the present invention. In this embodiment aperture 500 and second aperture 512 are different sizes. It may be desirable to have the aperture 500 and the second aperture 512 be different sizes in order to expose a preferred surface area of the respective electrode. Additionally, in FIG. 5E, the second reactive chemistry 502 is included, fills the aperture 500 and is directly in contact with the working conductor 202a. As illustrated, the first reactive chemistry 206 covers the opening 112 and overlaps the insulation 201. Additionally, the first reactive chemistry 206 covers both the working conductor 202a and the second reactive chemistry 502.

On the sensor B-side 100b, the CRC 202b includes the second aperture 512 where the third transport material 210 spans the width of the sensor from sensor edge 108a to sensor edge 108b while covering the insulation 201, the CRC 202b and filling the second aperture 512. As illustrated, the third transport material 210 also partially fills the aperture 500 and abuts or is directly in contact with the second reactive chemistry 502. This enables fluid surrounding the sensor to be transported through the third transport material 210 from the sensor B-side 100b to the second reactive chemistry 502. This enables the second reactive chemistry 502 supply cofactor to the reaction generated between the analyte of interest transported through the third transport material 210 on the A-side 100a to the reactive chemistry 206.

FIG. 5F is an exemplary illustration of a plurality of conductors and insulation that can be configured via the selective application of reactive chemistries to enable detection or sensing of a plurality of analytes of interest, in accordance with embodiments of the present invention. The embodiment illustrated in FIG. 5F is intended to illustrate the flexibility of the electrode configurations discussed throughout the disclosure. Included in FIG. 5F is a first electrode 520, a second electrode 522, and third electrode 524 and a fourth electrode 526. As illustrated, the first electrode 520 includes apertures 500. However, in other embodiments, any of the other electrodes 522, 524, or 526 may also be configured with apertures. In some embodiments, at least one of the electrodes 520, 522, 524 and 526 may be configured to be a combined counter reference electrode. This leaves three electrodes that can be configured to operate as three separate and independent working electrodes, where each can be configured to detect a different or same analyte of interest. For example, with a shared counter reference electrode, various configurations of the sensor assembly show in FIG. 5F can detect or measure either three or two analytes of interest (where one analyte is detected using two working electrodes), or alternatively, a single analyte of interest via three separate and independent working electrodes.

The ability to configure the various electrodes further illustrates that at least two of the electrodes 520, 522, 524, and 526 may be utilized as a discrete counter electrode and a discrete reference electrode. With a discrete counter electrode and a discrete reference electrode, the remaining two electrodes may be configured to measure concentrations of different or same analytes of interest. Exemplary non-limiting embodiments include a configuration where the electrode 524 is configured as a counter electrode and electrode 526 is configured as a reference electrode. This leaves electrode 520 and electrode 522 to be configured as working electrodes for either a first and second analyte of interests or a single analyte of interest.

In still another embodiment, electrode 520 may be configured as a combined counter reference electrode leaving electrodes 522, 524, and 526 to be configured as working electrodes. In still other embodiments, electrode 520 may be split into two separate electrodes thereby enabling a configuration of five independent electrodes. In one configuration utilizing five electrodes, one electrode is used as a shared counter/reference electrode that operates between two working electrodes to support anodic electrochemical reactions. The remaining two electrodes are configured as a third working electrode and a second counter/reference electrode to support cathodic electrochemical reactions.

For additional information and details regarding embodiments of a working electrode having an aperture through the working conductor, see U.S. Pat. No. 11,298,059 to Shah et al. the contents of which are hereby incorporated by reference in their entirety.

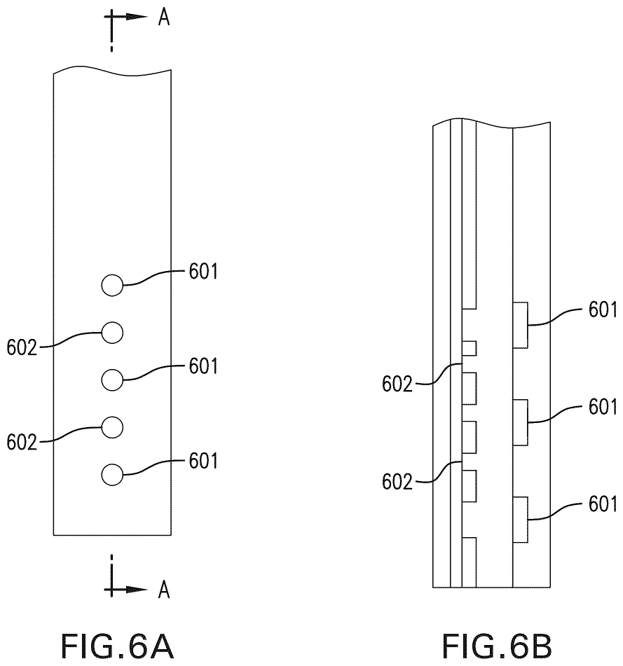

FIGS. 6A and 6B are exemplary illustrations of a sensor configured with sensing and non-sensing electrodes in order to control mass transfer gradients in diffusion fields primarily parallel to the electrode surface, in accordance with embodiments of the present invention. FIG. 6A is a top view of a sensor that illustrates an array of openings 601 and 602 in an insulation layer covering a working conductor. In preferred embodiments, a reactive chemistry containing an enzyme that is responsive to the analyte of interest is selectively applied to some of the openings formed on the working conductor. Portions of the working conductor that receive the selectively applied reactive chemistry become sensing electrodes while the portions of the working conductor that do not receive reactive chemistry are non-sensing electrodes.

In some embodiments, glucose oxidase is a component within the first reactive chemistry in order to enable the sensing electrodes to generate hydrogen peroxide in the presence of the analyte of interest, glucose. Thus, while the sensing electrodes generate hydrogen peroxide in the presence of the analyte of interest, the non-sensing electrodes function as sinks to decompose hydrogen peroxide generated by the sensing-electrodes.

In many embodiments, additional features or elements can be included, added or substituted for some or all of the exemplary features described above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. An analyte sensor, comprising:
a working conductor having an electrode reactive surface;
a first reactive chemistry being responsive to a first analyte;
a first transport matrix that includes a first transport material and a mitigation compound, the first transport material enables flux of the first analyte to the first reactive chemistry; and
a second transport material disposed over and configured to enable transport of a reactant to the first reactive chemistry,
wherein the first reactive chemistry does not contact the electrode reactive surface while at least partially overlapping a portion of the electrode reactive surface.

2. The analyte sensor described in claim 1, wherein flux of the first analyte to the first reactive chemistry is through an exposed lateral edge of the first transport matrix.

3. The analyte sensor described in claim 2, wherein the second transport material does not extend to the exposed lateral edge of the first transport matrix.

4. The analyte sensor described in claim 3, wherein the first transport matrix is directly in contact with the working conductor.

5. The analyte sensor described in claim 4, wherein the reactive chemistry is directly in contact with the first transport matrix.

6. The analyte sensor described in claim 5, wherein the second transport material is directly in contact with the first transport matrix and the reactive chemistry.

7. The analyte sensor described in claim 1, wherein the mitigation compound has a negative electrical charge.

8. A method to expose an electrical conductor that is encapsulated within an electrical insulator, the method comprising:
removing electrical insulator over a portion of a top of the electrical conductor to create a window within the electrical insulator, the window having a window edge, the window edge overlapping a portion of the top of the electrical conductor.

9. The method of claim 8, further comprising removing additional electrical insulator to expand the window, the removal of additional electrical insulator resulting in the window edge being aligned with a conductor edge.

10. The method of claim 8, further comprising, removing additional electrical insulator to expand the window, the removal of additional electrical insulator resulting in an insulator recess that extends away from the conductor.

11. The method of claim 10, wherein the insulator recess is mostly coincident with the top of the electrical conductor.

12. The method of claim 10, wherein the insulator recess is mostly coincident with a bottom of the electrical conductor.

13. The method of claim 10, wherein the insulator recess is below the bottom of the electrical conductor.

14. A working electrode within an electrochemical sensor assembly, comprising:
a multilayer structure having an A-side and a B-side, the A-side including a first insulation layer, a conductive layer adjacent to the first insulation layer, a via that traverse through the multilayer structure from the A-side to the B-side, the A-side further having a first reactive chemistry disposed over and in contact with a portion of the first insulation layer, the first reactive chemistry further being in disposed over and in contact with the conductive layer and partially filling the via to define a reactive via having a reactive area;
the B-side including a second insulation layer, the via traverses through second insulation layer and a second reactive chemistry partially filling the via from the B-side,
wherein the first reactive chemistry within the via prevents the second reactive chemistry from being in contact with the conductive layer.

15. The working electrode of claim 14, wherein the first reactive chemistry reacts with the analyte of interest and the second reactive chemistry includes a cofactor required by the first reactive chemistry.

16. The working electrode of claim 15, further comprising:
a first transport material disposed over the A-side, the first transport material extending across the sensor assembly to cover the first insulation layer and the reactive chemistry,
wherein the first transport material enables minimally encumbered transport and flux of an analyte of interest.

17. The working electrode of claim 16, further comprising:
a second transport material disposed over the A-side, the second transport material at least partially covering the first transport material,
wherein the second transport material is impervious to the analyte of interest.

18. The working electrode of claim 16, wherein the first transport material is applied to the B-side.

19. The working electrode of claim 18, wherein the first transport material on the B-side encapsulates the second reactive chemistry.

20. The working electrode of claim 18, wherein the first transport material applied to the B-side extends across the sensor assembly.
